(19) United States
(12) Patent Application Publication
(10) Pub. No.: US 2013/0317377 A1
(43) Pub. Date: Nov. 28, 2013

(54) Generative Model-Driven Resource-Efficient Monitoring in Body Sensor Networks

(71) Applicants: University of Washington through its Center for Commercialization, Seattle, WA (US); Arizona Board of Regents, a body corporate of the State of Arizona, acting for and on behalf of, Scottsdale, AZ (US)

(72) Inventors: Sandeep Gupta, Phoenix, AZ (US); Ayan Banerjee, Tempe, AZ (US); Sidharth Nabar, Seattle, WA (US); Radha Poovendran, Seattle, WA (US)

(73) Assignees: University of Washington through its Center for Commercialization, Seattle, WA (US); Arizona Board of Regents, a body corporate of the State of Arizona, acting for and on behalf of, Scottsdale, AZ (US)

(21) Appl. No.: 13/901,442

(22) Filed: May 23, 2013

Related U.S. Application Data
(60) Provisional application No. 61/650,560, filed on May 23, 2012.

(51) Int. Cl. A61B 5/0452; A61B 5/044; A61B 5/00

(52) U.S. Cl. CPC A61B 5/04525; A61B 5/0006; A61B 5/044
USPC 600/515

(57) ABSTRACT

Body sensor networks (BSNs) and methods for monitoring an electrocardiogram using such BSNs include a base station that generates an ECG model and an output ECG signal for displaying on a display device, and a sensor platform in electrical communication with the base station. The sensor platform may be configured to receive a sensed ECG signal from one or more sensors, receive an instance of the ECG model, and produce a model ECG signal from the instance. The sensor platform compares the sensed ECG signal to the model ECG signal and, if a deviation of the sensed ECG signal from the model ECG signal exceeds a threshold, transmits deviation data describing the deviation to the base station module. The sensor platform module does not transmit any data to the base station if there is no such deviation.

### CROSS-REFERENCE TO RELATED APPLICATIONS

This Application claims priority to U.S. Provisional Patent Application Ser. No. 61/650,560 filed May 23, 2012, incorporated by reference herein in its entirety.

### STATEMENT REGARDING FEDERALLY SPONSORED RESEARCH

Research described in this application was partially funded by ARO MURI Grant Number W911NF0710287 and NSF grant CT-0831544. The government has certain rights in this invention.

### FIELD OF INVENTION

This invention relates to electrocardiogram monitoring. In particular, this invention relates to body sensor networks that record and transmit an electrocardiogram with reduced data storage and energy consumption requirements.

### BACKGROUND OF THE INVENTION

An electrocardiogram (ECG) is a time-varying signal representing the electrical activity of the heart, and is an effective, non-invasive diagnostic tool for cardiac monitoring. Recently, several systems have been developed for continuous, remote ECG monitoring using Body Sensor Networks (BSNs). Such systems typically consist of a wireless, battery-operated, body-worn sensor that collects ECG data and transmits it to a gateway device such as a smartphone. The gateway reports this data over the internet to a remote base station, which is typically a hospital server or caregiver's computer. Such remote monitoring allows collection of data during a person's daily routine and enables early detection of conditions such as tachycardia or angina. Further, the availability of continuous long-term data can help identify gradual, long-term trends in the cardiac health of at-risk patients.

A key challenge in BSN-based ECG monitoring is the large volume of data collected by the sensor in a short time interval. For example, at a clinically-recommended sampling rate of 250 Hz and resolution of 12 bits/sample, more than 2 KB of data is collected within 6 seconds. Local storage of this data on the sensor or the gateway device is impractical due to storage limitations. Further, wireless transmission of this data consumes significant power at the energy-constrained sensor. At the same time, the quality and continuity of the reported ECG signal must be maintained at the base station to allow effective investigation and diagnosis by a physician.

Most current attempts to address these key challenges are based on data compression, where the sensed ECG data is compressed before transmission. Several techniques based on wavelets, Huffman coding and priority-based encoding have been proposed in literature. Unfortunately, known compression schemes need to continuously transmit data, thus limiting their energy savings. In one alternative approach, a set of features is extracted from the sensed ECG and used for classification. The preprocessing and pattern recognition workload is transferred to local nodes close to the ECG leads to reduce transmission energy consumption. This scheme, however, does not provide a complete sensed ECG signal at the base station and thus its value for diagnosis is limited. Another compressive sensing approach has been proposed for ECG monitoring, which uses the sparsity of the ECG signal in specific wavelet transformations to reduce sampling rate. However, reconstruction of the received signal is complex and strongly depends on error-free transmission of all coefficients.

### SUMMARY OF THE INVENTION

The present invention provides methods for monitoring an electrocardiogram. In one embodiment, the methods include receiving a sensed ECG signal from one or more sensors configured to collect the sensed ECG signal from the patient, comparing the sensed ECG signal to a model ECG signal, and, if a deviation of the sensed ECG signal from the model ECG signal exceeds a threshold, transmitting deviation data describing the deviation to a base station.

In another embodiment, the methods include receiving, at a sensor platform, a sensed ECG signal from one or more sensors configured to collect the sensed ECG signal from the patient and comparing, at the sensor platform, the sensed ECG signal to a model ECG signal. If a deviation of the sensed ECG signal from the model ECG signal exceeds a threshold, the methods may further include transmitting, from the sensor platform, deviation data describing the deviation to a base station. The methods may further include generating, at the base station, an output ECG signal to be displayed on a display device, wherein the output ECG signal comprises the model ECG signal and, when deviation data is received, further comprises a modification to the model ECG signal.

The present invention further provides systems for monitoring an ECG. In one embodiment, the system is a body sensor network for monitoring an electrocardiogram of a patient. The body sensor network may include a base station comprising a base station module configured to generate an ECG model and to generate an output ECG signal for displaying on a display device, and a sensor platform in electrical communication with the base station. The sensor platform may have a sensor platform module configured to: receive a sensed ECG signal from one or more sensors attached to the patient and collecting the patient's ECG embodied in the sensed ECG signal; receive an instance of the ECG model and produce a model ECG signal from the instance; compare the sensed ECG signal to the model ECG signal; and, if a deviation of the sensed ECG signal from the model ECG signal exceeds a threshold, transmit deviation data describing the deviation to the base station module. The sensor platform module does not transmit the sensed ECG signal if there is no deviation of the sensed ECG signal from the model ECG signal exceeding the threshold.

### BRIEF DESCRIPTION OF THE DRAWINGS

FIG. 1 is a diagram of an ECG beat.

FIG. 2 is a diagram of an ECG body sensor network in accordance with the present disclosure.

FIG. 3 is a schematic diagram of a data reporting implementation within a sensor module and a base station module in accordance with the present disclosure.

FIG. 4 is a flow diagram of a method of reporting an ECG in accordance with the present disclosure.

FIG. 5 is a flow diagram of a training method in accordance with the present disclosure.

FIG. 6 is a flow diagram of a method of calculating morphology parameters in accordance with the present disclosure.

FIG. 7A is a diagram of a training model compared to a normal ECG beat in accordance with the present disclosure.

FIG. 7B is a diagram of a training model compared to an abnormal ECG beat in accordance with the present disclosure.

FIG. 8 is a flow diagram of operations of a sensor module in accordance with the present disclosure.

FIG. 9 is a flow diagram of a method of preprocessing a sensed ECG signal in accordance with the present disclosure.

FIG. 10 is a flow diagram of a method of detecting peaks in a sensed ECG signal in accordance with the present disclosure.

FIG. 11 is a flow diagram of a method of comparing interbeat parameters of a sensed ECG signal to a model ECG in accordance with the present disclosure.

FIG. 12 is a flow diagram of a method of comparing morphology parameters of a sensed ECG signal to a model ECG with a direct signal comparison approach in accordance with the present disclosure.

### DETAILED DESCRIPTION OF THE INVENTION

The following discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of embodiments of the invention. Skilled artisans will recognize the examples provided herein have many useful alternatives and fall within the scope of embodiments of the invention.

As used herein, the singular forms “a”, “an” and “the” include plural referents unless the context clearly dictates otherwise. “And” as used herein is interchangeably used with “or” unless expressly stated otherwise. All embodiments of the invention can be combined unless the context clearly dictates otherwise.

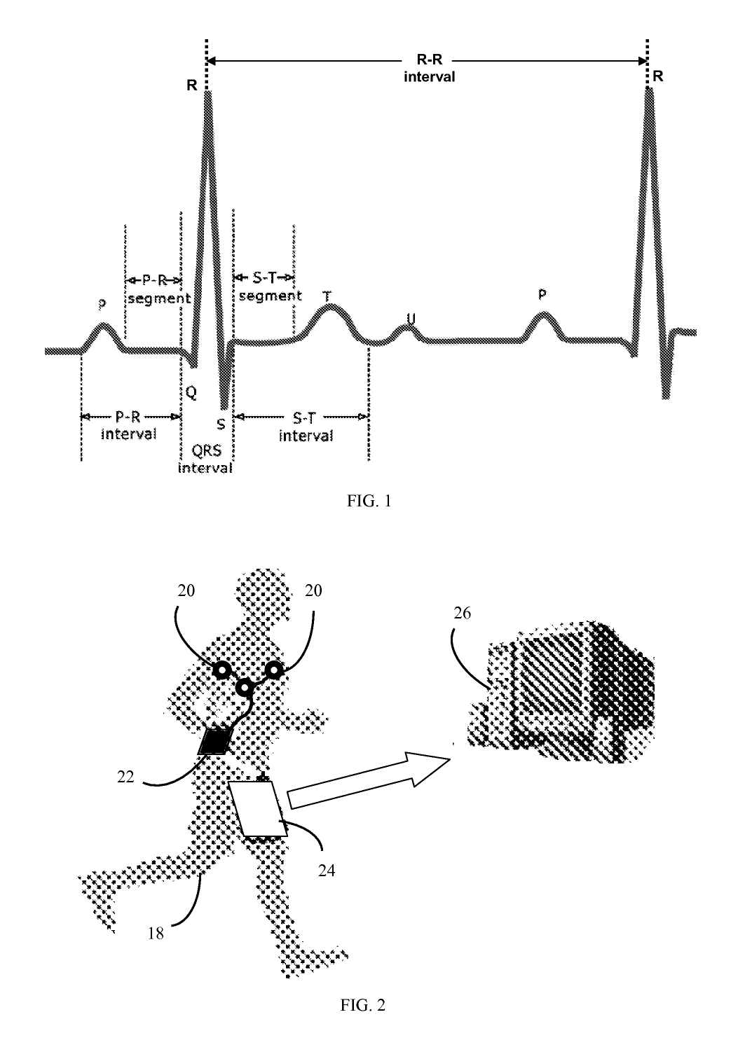

The ECG signal bas been extensively studied and used for cardiac diagnosis. The basic unit of an ECG is a beat, and its shape is referred to as the ECG morphology. Referring to FIG. 1, a single beat consists of P, Q, R, S and T waves, with a U wave present in some cases. The R wave is typically the most prominent and easy to identify in a beat. The Q, R and S waves are often jointly considered in a single complex, called the QRS complex. The shape, amplitude and relative locations of the constituent waves are key features of an ECG, and are referred to herein as morphology features. These features vary across individuals, but are expected to remain fairly stable for a given person, in the absence of pathological conditions.

The distance between two consecutive R peaks is called the R-R interval, and its reciprocal gives the instantaneous heart rate. Even in a healthy person, the R-R interval varies across beats due to several physiological factors. This variation is described using temporal features such as mean and standard deviation of heart rate, and spectral features such as Low Frequency/High Frequency (LF/HF) ratio. The temporal and spectral features of the ECG are referred to herein as interbeat features.

ECG is inherently a low amplitude electrical signal and is often corrupted by noise from various sources such as electrical mains, muscle noise and patient movement or respiration. As a result, the measured signal must be filtered as described below to extract the underlying ECG waveform. Among the constituent waves, the QRS complex can be extracted using computationally lightweight algorithms. The extraction of P and T waves, however, requires advanced filtering techniques that are computationally expensive to implement on sensors. Further, several conditions such as bradycardia, tachycardia, myocardial intimation and bundle branch block can be diagnosed from the QRS complex alone. As a result, some embodiments in accordance with the present disclosure may collect and analyze only the QRS complex of a set of ECG beats, to the exclusion of the other waves. In other embodiments, one or more of the P, Q, R, S, T, and U waves may be analyzed individually or collectively in order to obtain more complete diagnostic or condition-focused information.

The embodiments of the present disclosure may use a generative ECG model configured to produce synthetic ECG signals based on a set of input parameters. Some embodiments may use one or a combination of known dynamic model generators, such as ECGSYN. ECGSYN models an ECG signal as a point moving around a unit circle, and uses differential equations to describe its motion. The individual waves are modeled as Gaussian attractors/repellers placed at specific points on the circle. The inter-beat features of ECG are modeled using 3 parameters: hrmean, hrstd and lfhfratio, corresponding to mean heart rate, standard deviation of heart rate and LF/HF ratio respectively. For the morphology features, each wave is represented by 3 parameters: (a, b, θ), which determine its height, width and distance to R peak, respectively. For example, the Q wave is represented by the 3-tuple (a_{Q}, b_{Q}, and θ_{Q}).

Referring to FIG. 2, a BSN system model for sensing the ECG of a patient 18, processing the ECG, transmitting and storing data related to the ECG, and generating a visual representation of the ECG includes one or more sensors 20 in electrical communication with a sensor platform 22. The sensors 20 may be any suitable ECG sensor, such as a SHIMMER 3-lead wired or wireless sensor. The sensors 20 may be connected to the sensor platform 22 by wires, or may communicate wirelessly with the sensor platform 22. In some embodiments, the sensors 20 may be dumb sensors, in that the sensors 20 simply collect sensor data, specifically an ECG signal, and transmit the raw sensor data to the sensor platform 22. In other embodiments, the sensors 20 may be smart sensors equipped with a central processing unit (“CPU”) or microprocessor having sufficient computing resources to perform some of the ECG signal processing described below. A sensor 20 may be configured to collect sensor data in either integer or floating-point format, with integer-based implementation exhibiting improved memory footprint over floating-point. For example, integer implementation on the sensor platform may reduce the memory requirement of the sensor application from 9 KB down to 4 KB as compared to floating-point implementation.

The sensor platform 22 may be configured to receive data from the sensors 20 and to communicate data to a gateway device 24. In some embodiments, the sensor platform 22 may be a standalone device that transmits data through wired or wireless electrical connection to the gateway device 24. An example of such a sensor platform 22 is the SHIMMER Wireless Sensor Unit/Platform SH-SHIM-KIT-004, which is configured to transmit data via Bluetooth to a Bluetooth-enabled gateway device 24. In other embodiments, the sensor platform 22 may be a hardware or software module attached to or contained within the gateway device 24. The sensor platform 22 may comprise computing hardware and software, including a CPU, memory, data storage, and input/output terminals, having sufficient computing capacity to implement the sensor platform module described below. The sensor platform 22 may therefore receive raw or processed sensor data from the sensors and perform additional processing on the sensor data before transmitting data to the gateway device 24. In some embodiments, such as those embodiments implementing the methods described in detail below, the sensor platform 22 may receive a sensed ECG signal from the sensors, process the sensed ECG signal to produce deviation data, and transmit the deviation data to the gateway device 24.

The gateway device 24 may be configured to receive data from the sensor platform 22 and to communicate the data to a base station 26. The gateway device 24 may be any device suitable for receiving data transmitted by the sensor platform 22, which may be over a first communication network, and for transmitting the data to the base station 26 over a second communication network, which may be different from or may use different communication protocols or data security measures than the first communication network. In some embodiments, the gateway device 24 may be a personal mobile communication device, such as a smartphone. The gateway device 24 may communicate with the sensor platform 22 via Bluetooth, wired or wireless Local Area Network, or another limited-range wireless communication protocol or network. The gateway device 24 may communicate with the base station 26, which may be remote from the gateway device 24, via a cell network, a Wide Area Network, a telephone network, or another long-range data transfer network. The described communication system may use any suitable data encryption algorithm, username/password authentication, and other forms of data security to protect transmitted data.

In some embodiments, the base station 26 may be a computer, such as a personal computer, medical office or hospital server or mainframe, or another suitable computer for receiving data from the gateway device 24 and processing the data in order to display ECG information to a user, such as the patient or the patient's physician. The base station 26 may comprise computing hardware and software, including a CPU, memory, data storage, and input/output terminals, having sufficient computing capacity to implement the base station module described below. The base station 26 may therefore be configured to communicate with a body sensor network in order to receive a training ECG signal and to generate, distribute, and use a model ECG signal according to the present disclosure. In some embodiments, the base station 26 may be sufficiently robust to operate the ECGSYN dynamic model generator or another similar model generator.

Other embodiments in accordance with the invention may omit the gateway device 24. In such an embodiment, the sensor platform 22 may communicate the sensor data directly to the base station 26. The sensor platform 22 may be a standalone device as described above, or may be a hardware or software module attached to or contained within the base station 26. The base station 26 may be a personal mobile device such as a smartphone configured with Bluetooth or other data sharing technology, and further having a user interface for presenting ECG and receiving user input.

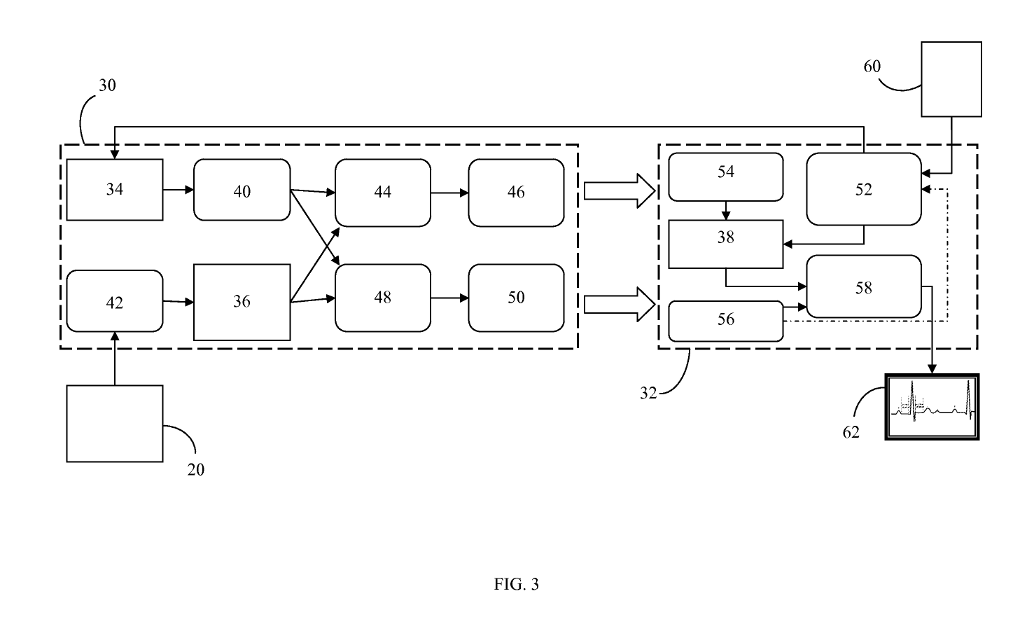

Referring to FIG. 3, a sensor platform module 30 may be implemented as hardware or software, or a combination thereof, on the sensor platform 22, and a base station module 32 may be implemented as hardware or software, or a combination thereof, on the base station 26. As described above, the sensor platform module 30 and base station module 32 may be on the same or different physical devices. Data may be transmitted between the modules 30, 32 via wired or wireless connection, or a combination thereof. For example, the sensor platform module 30 may be initialized as described below through temporary wired attachment of the sensor platform 22 to the base station 26 in order to receive the initial or updated model or to offload stored data from the sensor platform 22 to the base station 26. During operation of the BSN, however, the sensor platform 22 transmits data wirelessly to the base station 26 in this example.

The base station module 32 may be configured to train a dynamic model ECG based on input from one or more training sensors 60 attached to the patient. Prior to deploying the BSN for the patient, the base station module 32 may receive training data comprising an ECG signal recorded by the training sensors 60. At node 52, model learning takes place, wherein the base station module 32 may execute a stored dynamic model generator, such as ECGSYN. The model generator takes the training data as input parameters to generate the model ECG. The base station module 32 may be configured to distribute the model ECG to any device in the BSN that uses the model ECG for processing. FIG. 3 shows a base station instance 38 of the ECG model, and a sensor platform instance 34 of the ECG model. The sensor platform instance 34 may be a “lightweight” embodiment of the ECG model, in that the sensor platform instance 34 includes fewer datapoints or is otherwise streamlined in comparison to the base station instance 38. This allows the ECG model to consume less resources, with respect to both storage requirements and for data comparison purposes as described below. The complexity of the sensor platform instance 34 may be selected to maximize efficient use of computing resources without compromising accuracy. In particular, in some embodiments the sensor platform, which may not have a training module, implementsthe ECG model using integer point arithmetic which provides requisite precision without needing the floating point support.

During regular operation, the sensor platform module 30 may use the sensor platform instance 34 to, at node 40, generate a model ECG signal. The sensor platform module 30 may, at node 42, receive a sensed ECG signal from the sensors 20. Where the sensed ECG signal is a raw signal, the sensor platform module 30 may deliver the sensed ECG signal to a pre-processing module 36 that may be configured to format the sensed ECG signal for comparison to the model ECG signal as described in detail below. At nodes 44 and 48, the sensor platform module 30 may compare the sensed ECG signal to the model ECG signal. Specifically, at node 44 the sensor platform module 30 may compare the morphology features of the two ECG signals, and at node 48 the sensor platform module 30 may compare the interbeat features of the two ECG signals. If the ECG signals match within one or more predefined thresholds, the sensor platform module 30 may not report any data to the base station module 32. Conversely, if the sensed data deviates from the model beyond the thresholds, the sensor platform module 30 may transmit one or more data updates to the base station module 32. Specifically, at node 46 the sensor platform module 30 may transmit one or more deviation values, and at node 50 the sensor platform module 30 may transmit a portion of the sensed ECG signal as raw data. These comparisons and transmissions are described in detail below.

Returning to the base station module 32, at node 58 the base station module 32 may use the base station instance 38 of the ECG model to generate an output ECG and transmit the output ECG to a display device 62. The base station instance of the ECG model may be updated at node 54 using received deviation values as input parameters to update the corresponding parameters of the ECG model. Node 58 may further include temporally aligning the ECG model with the sensed ECG signal received at node 56 as raw data. Thus, while no data is received from the sensor platform module 30, the base station module 30 assumes that the ECG of the patient is close to the ECG model and uses the model to generate a synthetic ECG signal, which is used at the display device 62 to represent the patient's ECG. When data is received from the sensor platform module 30, it may be directly recorded as the patient's ECG to modify the representation of the patient's ECG at the display device 62. The sensor platform module 30 may be configured to periodically transmit connection acknowledgement messages to the base station module 32 so that the base station module 32 may differentiate between periods of conforming ECG signal (i.e. no data sent) and device or network failure.

Several features of ECG data, such as mean heart rate and the LF/HF ratio, vary over time with activities such as sleeping, walking and exercise. As a result, a single, static ECG model may not effectively represent a patient's ECG over extended periods of time. For effective operation, the present BSN may dynamically update the ECG model as the patient's ECG changes. Since the deviation of sensed ECG from model-based values is first detected at the sensor platform module 30, the sensor platform module 30 may trigger the modifications to the ECG model through communication of data to the base station module 30. This may be achieved on the computationally-limited sensor platform 22 using one or a combination of feature updates and raw signal updates. For feature updates, interbeat features of the sensed ECG signal (e.g. mean heart rate) may be calculated from sensed data, and when these values change significantly, the sensor platform module 30 may update the corresponding parameters of its own instance 34 of the ECG model, and further may report the calculated deviation values to the base station module 32 for updating the base station instance 38 as described above. For raw signal updates, when the morphology of the patient's ECG deviates from the ECG model, the sensor platform module 30 may send the raw sensed data to the base station module 32. Based on received data, the base station module 32 may derive new parameter values for the ECG model using the model learning functionality at node 52. These values may be communicated to the sensor platform module 30 for updating the sensor platform instance 34.

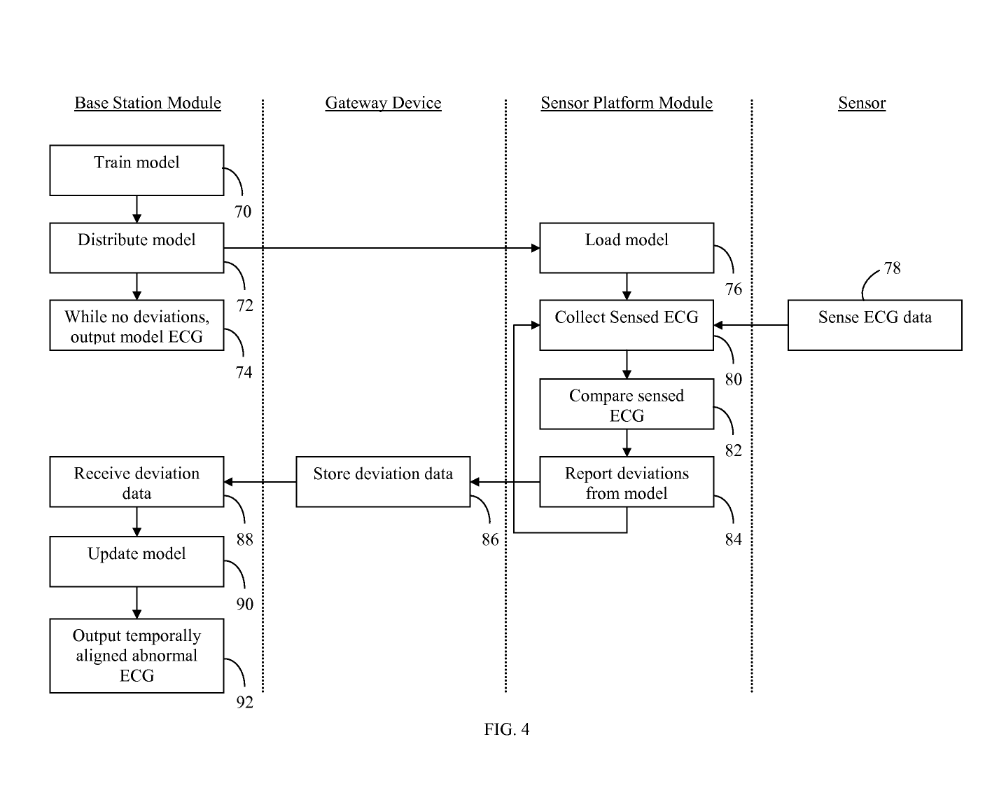

FIG. 4 illustrates a method of using a BSN to monitor ECG of a patient according to the present disclosure. At step 70, the base station module 32 may train the ECG model. At step 72, the base station module 32 may distribute the ECG model to the sensor platform module, which loads the ECG model at step 76. When the base station 26 and sensor platform 22 are configured with the ECG model, patient monitoring may commence. While no data describing deviations from the ECG model is received, the base station module 32 generates an output ECG signal that comprises the ECG model at step 74. Meanwhile, the sensors 20 on the patient sense the patient's ECG at step 78 and transmit the sensor data, comprising a sensed ECG signal, to the sensor platform 22. At step 80, the sensor platform module 30 collects the sensed ECG signal and, at step 82, compares the sensed ECG signal to a model ECG signal of the ECG model. If there are any deviations in the sensed ECG signal from the model ECG signal, at step 84 the sensor platform module 30 reports the deviations. However, if there are no deviations beyond preset thresholds, described further below, the sensor platform module 30 does not transmit any data. By defining suitable thresholds for the comparison between the sensed and model-generated ECG, a large fraction of data transmission at the sensor can be suppressed, thus significantly reducing sensor energy consumption, These threshold values can be specified by the physician based on the application requirements as well as the patient's age, lifestyle and health condition. Further, they can be adjusted over time to accommodate a tradeoff between data accuracy and communication energy.

The deviations may be reported first to the gateway device 24, which stores the deviations at step 86. The present method provides reduced ECG data size for storage by representing ECG using model parameters instead of data samples. For example, for a time interval denoted [t_{A}, t_{B}], if the patient's ECG follows the ECG model with parameter values [p₁, p₂, . . . P_{N}], the data can be stored in a table or database as: “[t_{A}, t_{B}]:[p₁, p₂, . . . P_{N}]”. These values can be used at a later time as inputs to the ECG model to regenerate the corresponding ECG data. This representation significantly reduces data size, and can enable local storage of ECG data on a resource-limited device, such as the patient's smartphone, which is not feasible with direct storage of sample values. The deviation data may also or alternatively be stored on the sensor platform 22 or base station 26. At step 88, the base station module 32 may receive the deviation data and use the deviation data as input parameters to update the ECG model at step 90. At step 92, the base station module may temporally align any abnormal ECG signal with the model ECG signal and create a modified output ECG signal for displaying the abnormal ECG.

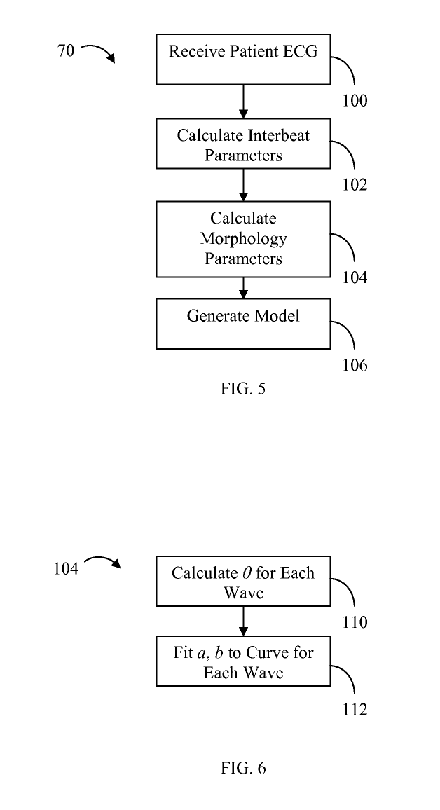

Referring to FIG. 5, the model learning function takes a real ECG signal as input, and generates a set of suitable input parameters for ECGSYN. The suitable input parameters may include interbeat parameters describing the interbeat features, and morphology parameters describing the morphology. In one embodiment of the step 70, training the model may include, at step 100, receiving the patient's ECG, such as from one or more training sensors 60 as described above. At step 102, the interbeat parameters may be calculated from the patient's ECG. These parameters may include the parameters hrmean, hrstd and lfhfratio, corresponding to the mean heart rate, standard deviation of heart rate and LF/HF ratio features of ECG respectively. In one embodiment of calculating the LF/HF ratio, a set of 256 R-R interval values is obtained from the patient ECG data and the Power Spectral Density (PSD) of this set is computed. The Low Frequency (LF) and High Frequency (HF) components are then obtained by integrating the PSD over the ranges (0.04 Hz-0.15 Hz) and (0.15 Hz-0.4 Hz), respectively. The ratio between these components gives the value of the lfhfratio parameter. The hrmean and hrsrd values may be obtained by performing averaging and standard deviation calculations on a discrete set of about 60 R-R interval values.

At step 104, the morphology parameters may be calculated from the patient's ECG. These parameters may include the (a, b, θ) parameters for each of the P, Q, R, S, T, and U waves. In one embodiment where only the QRS complex is evaluated, to the exclusion of the other waves, only 9 paratneters (a_{Q}, a_{R}, a_{S}, b_{Q}, b_{R}, b_{S}, θ_{Q}, θ_{R}, θ_{S}) are used to represent the beat morphology. Referring to FIG. 6, at step 110 the θ for each wave may be calculated. In the QRS-only embodiment, θ_{Q} and θ_{S} are calculated using the distance of the R peak from the Q and S peaks, respectively, while θ_{R} is zero, by definition. For learning the remaining parameters (a_{Q}, a_{R}, a_{S}, b_{Q}, b_{R}, b_{S}), a curve fitting approach may be used at step 112. A set of initial values for these parameters may be obtained by solving a system of linear equations using a number of points on the ECG signal equal to the number of parameter values to be obtained (six in the present example). Starting with these initial values, a least squares curve fitting function may adjust the values until the noise floor is reached.

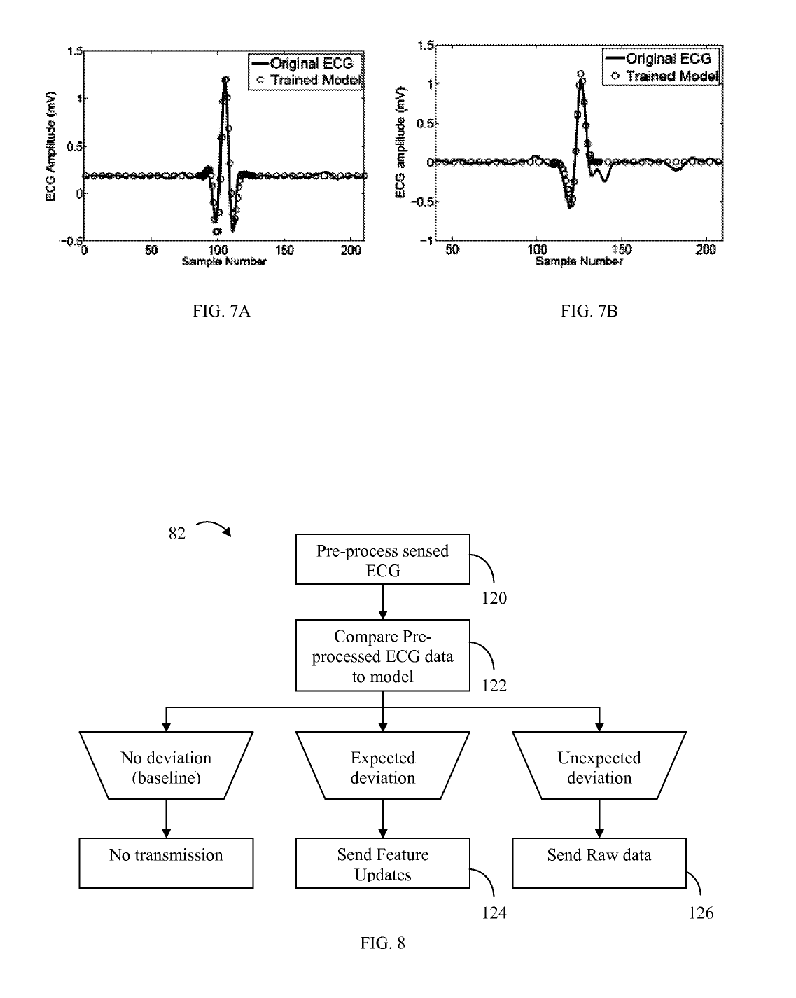

Thus, the interbeat and morphology parameters are learned from the patient's ECG and used to generate a matching synthetic ECG. The morphology of ECG may depend on the lead configuration of the sensors, and may vary across patients. Hence, the data used for learning the model should be obtained from the intended user of the system, and using the same lead configurations for training sensors 60 that are used for sensors 20. Referring back to FIG. 5, the obtained parameters may be used as input parameters for the dynamic model generator, such as ECGSYN, to generate the model at step 106. FIGS. 7A and 7B illustrate example fits of the model ECG signal, generated using the extracted parameters, to the patient's ECG signal collected by the training sensors 60, where FIG. 7A is a fit against a normal ECG and FIG. 7B is a fit against an ECG from a patient showing congestive heart failure.

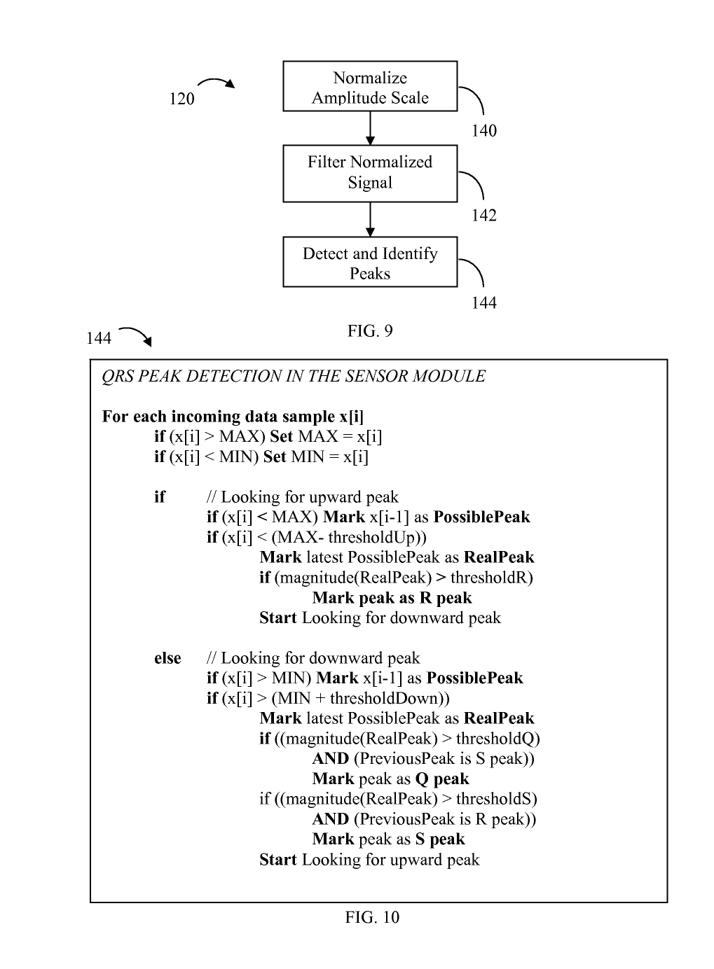

Referring to FIG. 8, the step 82 of comparing the sensed ECG signal to the model ECG signal may include, at step 120, pre-processing of the sensed ECG signal to convert it into a format suitable for comparison with the stored model. Referring to FIG. 9, preprocessing may include operations such as scaling, filtering, and peak detection. Example implementation details for each of these operations are as follows:

1) Scaling (step 140): the amplitude of the sensed ECG signal is highly dependent on the sensor 20 hardware and the ECG lead configuration of the sensor 20. To ensure an accurate comparison between the sensed ECG signal and the model ECG signal, both signals may be converted to a normalized, device-independent scale. This is achieved by linearly scaling each signal to a maximum of 1.2 mV and minimum of −0.4 mV.

2) Filtering (step 142): the sensed ECG signal is typically noisy, and may be filtered to remove the noise. For extracting the QRS complex, a passband of 5-12 Hz may be achieved by cascading lowpass and highpass filters with cutoff frequencies at about 5 Hz and about 12 Hz, respectively. For low computational overhead, a Finite Impulse Response (FIR) filter design of 6 taps and order 32 may be used.

3) Peak Detection (step 144): measuring ECG features such as R-R intervals or QRS complex width requires the identification of Q, R, and S peaks. FIG. 10 illustrates, in pseudocode, an algorithm for performing this peak detection at low computational overhead. This algorithm detects all the positive and negative peaks in a signal, and then imposes a relative threshold on the amplitude to qualify peaks as Q, R, and S. Further, false positives are reduced by imposing conditions based on the previous peak detected. For example, for a negative peak to be declared as ‘S’, the previous peak must be an R peak.

Referring again to FIG. 8, at step 122, the sensed ECG signal obtained from preprocessing may be compared to the model ECG signal. Such a comparison may be performed in two ways: the sensed ECG signal can be directly compared to the model ECG signal; or, a set of representative feature values can be extracted from each signal and these feature values can be compared. The feature comparison approach is more accurate for noisy measurements but incurs computational overhead for the calculation of the feature values. The feature values for interbeat features (mean and standard deviation of heart rate, and the LF/HF ratio) may be calculated at low computational cost, and so the feature comparison approach may be used for comparing interbeat features of the two signals. On the other hand, calculating the feature values of morphology features may be too resource-consuming because it requires a curve fitting approach. For devices where curve fitting calculations are not feasible or efficient, the direct signal comparison approach may be used to compare the ECG morphologies of the two signals.

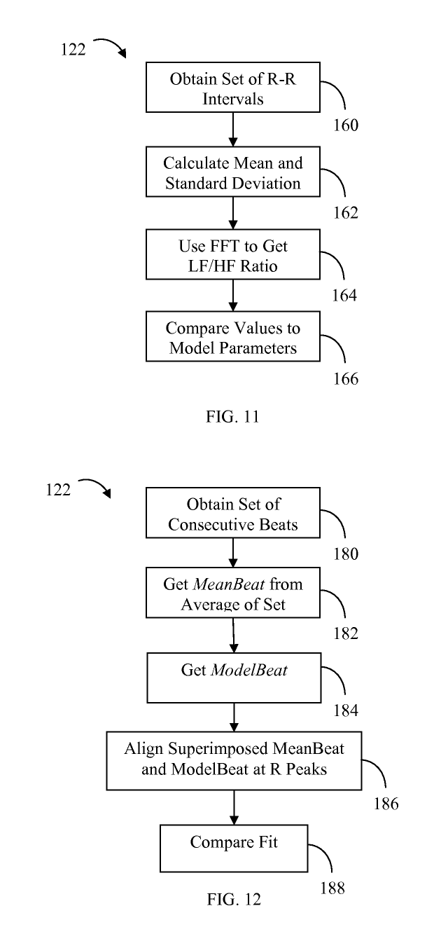

Referring to FIG. 11, where comparing the sensed ECG signal to the model ECG signal includes performing feature value calculations, the mean and standard deviation of the heart rate within the sensed ECG signal are obtained by, at step 160, obtaining a discrete set, such as 30, of consecutive R-R intervals and, at step 162, calculating the mean and standard deviation of the set of R-R intervals. The LF/HF ratio may be calculated as described above. Alternatively, to optimize computation speed and power consumption, a Fast Fourier Transform (FFT) configured particularly for performing in-place computations within the sensor platform module 30 may be used at step 164 to obtain the LF/HF ratio. Once these calculations are complete, at step 166 the calculated feature values are compared to model parameter values hrmean, hrstd and lfhfratio, respectively.

Referring to FIG. 12, where comparing the sensed ECG signal to the model ECG signal includes performing direct comparisons of the two signals, at step 180 a discrete set, such as 10, of consecutive beats within the sensed ECG may be obtained. At step 182 a sample, representative beat, referred to as meanBeat, for the sensed ECG signal may be obtained by averaging the set of consecutive beats. At step 184, a representative beat, referred to as modelBeat, for the model ECG signal may be obtained with a similar method to that of obtaining meanBeat, or the model generator may be configured to generate modelBeat. At step 186, the modelBeat and meanBeat are aligned by superimposing the respective R peaks, and at step 188 the fit is compared using a mean square error approach or another suitable comparison approach. The mean square metric may be advantageous because it captures shape as well as amplitude of the Q, R and S waves. The generation of modelBeat may be so computationally expensive that it is preferable to be performed only once, when new morphology parameter values are assigned. The generated modelBeat is then stored in memory for future use.

Referring again to FIG. 8, based on these comparisons, if the sensed ECG signal is found to deviate from the model, the sensor platform module 30 may report the deviation to the base station module 32. For interbeat features, if the mismatch between true feature values and corresponding model parameter values exceeds a pre-defined threshold, the sensor platform module 30 may update its own model parameters in the sensor platform instance 34. At step 124, the sensor platform module 30 may also report the feature update to the base station module 32. In the morphology comparison, if the error between meanBeat and modelBeat is above a specified threshold, the sensed ECG signal for the corresponding time interval (i.e. 10 beats as collected for determining meanBeat) may be sent to the base station module 32, at step 126. If the base station module 32 receives multiple such raw signal updates, it may retrain the morphology parameters of the ECG model, and may communicate the new values to the sensor platform module 30. Although such raw signal updates can incur significant data transmission at the sensor platform 22, the impact on overall energy consumption is minimal, since the ECG morphology of a person is not expected to vary much over time. Furthermore, current or future data compression schemes may be added to the present methods to reduce the data size in cases where the sensor platform 22 transmits raw data to the base station 26. This will help to further reduce energy consumption.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein. Various features and advantages of the invention are set forth in the following claims.

We claim:

1. A method for monitoring an electrocardiogram (ECG) of a patient, the method comprising:
receiving a sensed ECG signal from one or more sensors configured to collect the sensed ECG signal from the patient;
comparing the sensed ECG signal to a model ECG signal; and
if a deviation of the sensed ECG signal from the model ECG signal exceeds a threshold, transmitting deviation data describing the deviation to a base station.

2. The method of claim 1, further comprising transmitting no data other than the deviation data to the base station.

3. The method of claim 1, further comprising periodically transmitting an acknowledgment signal to the base state, and transmitting no data other than the deviation data and the acknowledgment signal to the base station.

4. The method of claim 1, wherein comparing the sensed ECG signal to the model ECG signal comprises one or more of: performing feature value calculations; and performing direct comparisons of the sensed ECG signal to the model ECG signal.

5. The method of claim 1, further comprising generating an output ECG signal, wherein the output ECG signal comprises the model ECG signal when no deviation data is transmitted, and wherein the output ECG signal comprises a modification to the model ECG signal when deviation data is transmitted, the modification being generated based on the deviation data.

6. The method of claim 1, wherein the deviation data comprises one or more feature updates.

7. The method of claim 6, further comprising updating the model ECG signal based on the one or more feature updates.

8. The method of claim 1, wherein the deviation data is raw data comprising a portion of the sensed ECG signal.

9. A method for monitoring an electrocardiogram (ECG) of a patient, the method comprising
receiving at a sensor platform a sensed ECG signal from one or more sensors configured to collect the sensed ECG signal from the patient;
comparing, at the sensor platform, the sensed ECG signal to a model ECG signal;
if a deviation of the sensed ECG signal from the model ECG signal exceeds a threshold, transmitting, from the sensor platform, deviation data describing the deviation to a base station; and
generating, at the base station, an output ECG signal to be displayed on a display device, wherein the output ECG signal comprises the model ECG signal and, when deviation data is received, further comprises a modification to the model ECG signal.

10. The method of claim 9, wherein comparing the sensed ECG signal to a model ECG signal comprises performing calculations of one or more feature values of the sensed ECG signal, comparing the one or more feature values to one or more corresponding model parameter values of the model ECG signal, and generating the deviation data comprising any of the one or more feature values that deviates from the corresponding model parameter values beyond the threshold.

11. The method of claim 10, further comprising updating an ECG model, from which the model ECG signal is derived, based on the deviation data.

12. The method of claim 9, wherein comparing the sensed ECG signal to a model ECG signal comprises:
obtaining a set of consecutive beats from the sensed ECG signal;
calculating a representative beat for the sensed ECG signal, comprising the average of the set of consecutive beats;
directly comparing the representative beat for the sensed ECG signal to a representative beat for the model ECG signal; and
generating the deviation data as raw data comprising either or both of the set of consecutive beats and the representative beat for the sensed ECG signal.

13. The method of claim 12, wherein the modification to the model ECG signal comprises an abnormal ECG signal generated using the deviation data.

14. The method of claim 9 further comprising periodically transmitting, from the sensor platform to the base station module, an acknowledgment signal, wherein the sensor platform does not transmit any data to the base station module other than the deviation data and the acknowledgement signal.

15. The method of claim 9, further comprising training, at the base station, an ECG model from which the model ECG signal is derived, the training comprising:
receiving a training ECG from the patient;
calculating one or more interbeat parameters from the training ECG;
calculating one or more morphology parameters from the training ECG; and
generating the ECG model using the interbeat parameters and the morphology parameters as inputs.

16. The method of claim 15, further comprising distributing the ECG model from the base station to the sensor platform.

17. A body sensor network for monitoring an electrocardiogram of a patient, the body sensor network comprising:
a base station comprising a base station module configured to generate an ECG model and to generate an output ECG signal for displaying on a display device; and
a sensor platform in electrical communication with the base station, the sensor platform comprising a sensor platform module configured to:
receive a sensed ECG signal from one or more sensors attached to the patient and collecting the patient's ECG embodied in the sensed ECG signal;
receive an instance of the ECG model and produce a model ECG signal from the instance;
compare the sensed ECG signal to the model ECG signal; and
if a deviation of the sensed ECG signal from the model ECG signal exceeds a threshold, transmit deviation data describing the deviation to the base station module;
wherein the sensor platform module does not transmit the sensed ECG signal if there is no deviation of the sensed ECG signal from the model ECG signal exceeding the threshold.

18. The body sensor network of claim 17, wherein the sensor platform module compares the sensed ECG signal to a model ECG signal by:
performing calculations of one or more feature values of the sensed ECG signal;
comparing the one or more feature values to one or more corresponding model parameter values of the model ECG signal; and
generating the deviation data comprising any of the one or more feature values that deviates from the corresponding model parameter values beyond the threshold;
and wherein the base station module is configured to update the ECG model based on the deviation data.

19. The body sensor network of claim 17, wherein the sensor platform module compares the sensed ECG signal to a model ECG signal by:
obtaining a set of consecutive beats from the sensed ECG signal;
calculating a representative beat for the sensed ECG signal, comprising the average of the set of consecutive beats;
directly comparing the representative beat for the sensed ECG signal to a representative beat for the model ECG signal; and
generating the deviation data as raw data comprising either or both of the set of consecutive beats and the representative beat for the sensed ECG signal;
and wherein the output ECG signal comprises an abnormal ECG signal that is based on the deviation data.
